(19) United States
(12) Patent Application Publication
(10) Pub. No.: US 2025/0295499 A1
(43) Pub. Date: Sep. 25, 2025

(54) METHOD AND KIT FOR FUSING BONES AT A JOINT

(71) Applicant: TriMed, Inc., Santa Clarita, CA (US)

(72) Inventor: Robert J. Medoff, Kailua, HI (US)

(21) Appl. No.: 18/828,261

(22) Filed: Sep. 9, 2024

Related U.S. Application Data
(60) Provisional application No. 63/569,528, filed on Mar. 25, 2024.

(51) Int. Cl. A61F 2/46; A61F 2/30

(52) U.S. Cl. CPC A61F 2/4606; A61F 2002/30616

(57) ABSTRACT

A method of, and kit for, fusing a first end of a first bone to a first end of a second bone at a joint. An elongate body on an implant has an end that is directed into the first bone. A second end of the implant body is directed into the second bone. By exerting a force on the implant body through a drawing component through the opening at the second end of the second bone: a) a length of the implant body in the first bone can be drawn out of the first bone; and b) the second end of the implant can be moved within the second bone towards the second end of the second bone so that separate lengths of the implant body are stably embedded in both bones.

### BACKGROUND OF THE INVENTION

### Field of the Invention

This invention relates to bone fusion and, more particularly, to a method and kit for fusing bones at a joint using an intramedullary implant.

Background Art

Fusion of interphalangeal joints of the hand or foot are procedures performed for treatment of conditions of the interphalangeal joints, such as arthritis and deformity. There are different implants used in, and methods of, performing these procedures. Hammer toe correction of the lesser toes is one common example of interphalangeal fusion. In this surgery, the proximal interphalangeal joint between the proximal and middle phalanges of the toe is fused. Although interphalangeal fusion of both the proximal and distal interphalangeal joints of the toes and fingers, as well as the interphalangeal joints of the great toe and thumb are done, hammer toe correction will be used as an example herein throughout. This, however, is not meant to be restrictive. In addition, the invention can be practiced in the same manner with other bones as well, such as, but without limitation, fusion of the carpus, metacarpophalangeal joints, tarsus, or long bones.

To fuse the proximal interphalangeal joint of a toe, the ends of bones are prepared and held in apposition to allow osseous union to occur between the bones. Typically, the bone ends are prepared by removing any residual cartilage and shaping the ends to fit against one another over a substantial area. Commonly, the surfaces are cut into flat planes which are then apposed to guide the alignment of one bone relative to the other into a desired fused relationship. Alternatively, bone ends may be shaped into other types of surfaces such as apposing convex/concave surfaces, step cut surfaces, etc. Once the bone ends are prepared and apposed in the end relationship, an implant is typically used to maintain this desired relationship until fusion occurs.

It is known to use pins and wires as a simple means for fixation of two bones at a joint. Exemplary prior art structures and methods will be described below with reference to an exemplary joint, as shown in FIG. 1, between a proximal phalanx, identified herein as a first bone 10, and a middle phalanx, identified herein as a second bone 12.

It is known to extend a pin/wire from the end of the toe, across both the distal and proximal interphalangeal joints and into the proximal phalanx. The end of the pin/wire remains exposed at the tip of the toe.

One exemplary prior art method of fusing the first and second bones 10, 12 in FIG. 1 will now be described in greater detail with reference to FIGS. 2-6.

As seen in FIG. 2, a surface 14 at a first end 16 of the first bone 10 cooperates with a surface 18 at a first end 20 of the second bone 12 at a joint location at 22.

The surfaces 14, 18 are processed, from their starting FIG. 1 shape, to create flat configurations that facially confront with the surfaces apposed and the first and second bones 10, 12 in the desired end, fused relationship of FIGS. 3-6.

To maintain the first and second bones 10, 12 in the fused relationship, a K-wire/pin 24 is advanced in the direction of the arrow 26 in FIG. 2 through the second end 28 of the second bone 12 and fully through an intramedullary space 30 of the second bone 12 to be exposed beyond the surface 18, as shown in FIG. 2.

The first and second bones 10, 12 are then repositioned from the FIG. 2 relationship into the fused relationship by directing a protruding tip of a free end 32 of the K-wire/pin 24 through the surface 14 in alignment with an intramedullary space 34 of the first bone 10, thereby allowing the surfaces 14, 18 to assume the apposed relationship shown in FIGS. 3 and 4.

With the bones 10, 12 and K-wire/pin 24 in the relationship of FIGS. 3 and 4, the K-wire/pin 24 is advanced in the direction of the arrow 36 in FIG. 4, thereby causing an additional length of the K-wire/pin 24 to penetrate the intramedullary space 34 of the first bone 10, as seen in FIGS. 5 and 6, which is the final position for the K-wire/pin 24. The additional extension into the first bone 10 gives the required stability for the fused connection.

As seen also in FIGS. 5 and 6, a discrete length of the K-wire/pin 24 at 38 remains exposed beyond the second end 28 of the second bone 12.

With this method, the K-wire/pin 24 is typically left in place until sufficient healing has occurred, whereupon it is removed/withdrawn, which is facilitated by having the readily engageable exposed length 38 of the K-wire/pin 24. Typically, the fusion/healing process may take several weeks or even months.

As a result, the post-operative course is generally painful and very awkward for the patient since a normal shoe cannot be worn and the patient must keep the tissue area around the exposed length 38 of the K-wire/pin 24 clean and dry.

Further, K-wires/pins often catch on external objects, resulting in pain or even premature dislodgment. Further, there is an elevated level of risk of infection due to the chronically exposed open wound at the entry site at the tip of the patient's toe.

While this technique may be relatively inexpensive, it is associated with significant morbidity.

Another technique, as shown in FIG. 7, uses one or more K-wires/pins 24 which are extended between the bones 10, 12 angularly to the overall length thereof so as to penetrate both of the apposed surfaces 14, 18.

One of the limitations of this technique is that it requires an entry/exit hole that is relatively close to the surfaces 14, 18 and the angular direction of the K-wire(s)/pin(s) 24 creates the likelihood that there may be a secondary fracture to one or more of the bones 12, 14 near the juncture of the surfaces 14, 18. Even if fracture does not occur during the surgery, the bones 12, 14 may be locally weakened, which may compromise the integrity of the fixation and potentially lead to a later failure.

In FIG. 8, instead of using a K-wire/pin 24, one ore more small screws 40 may be directed into the bones 10, 12 in a fashion similar to the manner that the K-wire/pins 24 are implanted in FIG. 7-angularly disposed relative to the combined length of the bones 10, 12 and in proximity to the apposed surfaces 14, 18.

Since the screws 40 will generally have a larger diameter than the K-wire/pins 24, the above problems described relative to FIG. 7 are even more pronounced—compromised fixation and potential fracture as at exemplary weakened locations 42a, 42b.

Still further, crossing K-wire/pins 24 and screws 40 can place an open wound near the fusion site, increasing the risk of osteomyelitis.

Another known fusion technique is depicted schematically in FIG. 9. A plate 44 spans the first and second bones 10, 12 and is externally secured to the first and second bones 10, 12 through one or more fixation components 46 directed through the plate 44 into each of the first bone 10 and second bone 12.

Generally, the plates 44 are relatively bulky, potentially resulting in hardware irritation due to the prominence of the plate under the patient's skin.

In addition, screw purchase, typically relied upon when using the fixation components 46, can be compromised due to the relatively small size of the bones 10, 12 and the potential poor quality of one or both of the bones 10, 12.

Another known method of fusion is currently promoted by Paragon 28 as its “Hammer Tube System”. As shown schematically in FIG. 10, one form of this technique involves using an intramedullary implant 48 as between a corresponding first bone/proximal phalanx 10 and a second bone/middle phalanx 12. Since this implant 48 cannot be inserted from the end of the digit made up of the first bone 10 and the second bone 12, one end 52 of the implant 48 is initially directed into a receptacle 54 formed through the surface 14 and into the intramedullary space 34 of the first bone 10. Distraction of the second bone 12 is then performed in order to move the surface 18, that will ultimately appose the surface 14, sufficiently away from the surface 14 so that the exposed end 58 of the embedded implant 48 can be maneuvered up to and into a receptacle 60 formed through the surface 18 and into the intramedullary space 30 on the second bone 12. The second bone 12 is then guided over the implant 48 until the surfaces 14, 18 are pushed together.

Commercially available intramedullary implants, of the type shown at 48 in FIG. 10, have a primary longitudinal axis that may be linear or with one axis portion in a proximal section being at a slight angle to an axis portion at a distal section to position the proximal phalange/first bone 10 and middle phalanx 12 in a non-linear desired longitudinal alignment, within which they are fused. Implants of this type may be solid or have split arms that engage the endosteal surface of the bone.

These implants may be formed using a variety of materials, including but not limited to, metal, polyetheretherketone (PEEK), bioabsorbable material, carbon fiber, bone, or even combinations of these different materials, and others.

The surfaces of such intramedullary implants can also vary, from smooth cylindrical geometries, to those with ribs, ridges, threads, teeth, and ingrowth surfaces. These implants may also vary in terms of elasticity and other parameters.

However, despite the variability of these existing, intramedullary implant designs, a common feature is that techniques using these implants require distraction, of the surfaces that are placed in apposed relationship with the bones fused, sufficiently that with one end of the implant inserted into one of the bones, the other bone can be maneuvered to direct the other projecting end of the implant up to and into a receptacle prepared therefor. However, connective soft tissue spanning the surfaces at the joint will allow only limited amounts of distraction. The greater the length of the exposed implant to be directed into the middle phalanx, to achieve the desired degree of holding and stability, the more difficult it is to maneuver the middle phalanx to align the projecting implant end with the formed receptacle in the middle phalanx to allow seating of the other end of the implant in the middle phalanx. With longer projecting lengths of the implant, this maneuvering may be impossible without inflicting soft tissue damage. Because of this challenge, designers of these implants have defaulted to a relatively small projecting length of the embedded implant. As a result, the relatively limited length of the implant that penetrates the middle phalanx creates a short lever arm within the intramedullary canal. This may result in compromised fixation, greater motion between the implant and bone, and increased risk of non-union or disengagement of the end of the implant from its respective bone during the post-operative period.

In spite of the above known limitations, those in the medical industry have contended with the same given the lack of alternative designs of components and techniques that effectively overcome the noted prior art limitations.

### SUMMARY OF THE INVENTION

In one form, the invention is directed to a method of fusing first and second bones at a joint. The second bone has first and second ends. A first end of the first bone is at the first end of the second bone where the joint is formed. The method includes the steps of: obtaining an implant having a length between first and second ends; directing the implant into an intramedullary space of the first bone so that a first length portion of the implant resides within the intramedullary space of the first bone; and exerting a force on the implant through an opening at the second end of the second bone as an incident of which: a) a part of the first length portion of the implant within the intramedullary space of the first bone moves out of the intramedullary space of the first bone; and b) one of the first and second ends of the implant is caused to move one of: i) into the intramedullary space of the second bone; and ii) further within the intramedullary space of the second bone towards the second end of the second bone.

In one form, the step of directing the implant involves directing the first end of the implant into the intramedullary space of the first bone. The one of the first and second ends of the implant is the second end of the implant.

In one form, the step of exerting a force on the implant involves exerting a force through a component that extends through the opening at the second end of the second bone and engages with the implant at or adjacent the second end of the implant.

In one form, the step of exerting a force on the implant involves exerting a force through a component that extends through the opening at the second end of the second bone and engages with the implant at a location between the first and second ends of the implant.

In one form, the method further includes the step of modifying at least one of the first end of the first bone and the first end of the second bone to define cooperating surfaces at the first end of the first bone and the first end of the second bone that are brought together with the first bone and second bone in a fused relationship.

In one form, the step of modifying involves causing the cooperating surfaces at the first end of the first bone and the first end of the second bone to have substantially flat apposed surface configurations with the first bone and second bone in the fused relationship.

In one form, the method further includes the step of forming a receptacle for the implant in at least one of the first bone and the second bone.

In one form, the step of forming a receptacle involves forming a receptacle in each of the first bone and the second bone. With the first bone and second bone in a fused relationship, one part of the implant is frictionally maintained within the receptacle formed in the first bone and another part of the implant is frictionally maintained within the receptacle formed in the second bone.

In one form, the step of exerting a force involves exerting a force through a component made up of a suture that is connected to the implant and projects from the opening at the second end of the second bone.

In one form, the suture connects to the implant at a location between the first and second ends of the implant.

In one form, the method further includes the step of engaging the suture with the implant with the suture extending from the implant through the intramedullary space of the second bone so that a graspable part of the suture projects from the intramedullary space of the second bone through the opening at the second end of the second bone. The step of exerting a force involves exerting a force on the graspable part of the suture.

In one form, the method further includes the steps of: obtaining a suture engaging tool; directing a part of the suture engaging tool in a first direction through the opening at the second end of the second bone and the intramedullary space of the second bone and engaging the part of the suture engaging tool with the suture; and with the part of the suture engaging tool engaged with the suture, moving the part of the suture engaging tool oppositely to the first direction to cause the graspable part of the suture to project from the intramedullary space of the second bone through the opening at the second end of the second bone.

In one form, the method further includes the steps of obtaining a suture engaging tool, engaging the suture engaging tool with the suture, and directing the suture engaging tool through an opening at the first end of the second bone to thereby advance a part of the suture through the intramedullary space of the second bone to cause the graspable part of the suture to project from the intramedullary space of the second bone at the second end of the second bone.

In one form, the suture is connected to the implant by directing the suture through an opening in the implant and doubling the suture against itself to create a bight portion that bears against the implant as a force is exerted through the suture.

In one form, the step of exerting a force involves exerting a force through a drawing component that is extended through the opening at the second end of the second bone and the intramedullary space of the second bone and connected to the implant at or adjacent to the second end of the implant.

In one form, the drawing component is releasably connected to the implant through a connector on the drawing component that is releasably connected to a connector on the implant.

In one form, the connectors on the drawing component and implant are releasably threadably engaged.

In one form, the receptacle in the first bone is a bore in the first bone enlarging the intramedullary space of the first bone. The receptacle in the second bone is a bore in the second bone enlarging the intramedullary space of the second bone.

In one form, with the first bone and second bone in a fused relationship, an axis of the bore in the first bone is aligned with an axis of the bore in the second bone.

In one form, a diameter of the bore in the first bone is different than a diameter of the bore in the second bone.

In one form, the implant has a body with a non-uniform diameter between the first and second ends of the implant.

In one form, the diameter of the body tapers over a length of the body.

In one form, the implant has a body with a lengthwise axis and an external surface that extends around the lengthwise axis. The external surface has a non-circular shape as viewed along the lengthwise axis over at least a portion of the length of the implant. With the first bone and second bone in a fused relationship, the non-circular shape causes the implant to be keyed against turning relative to at least one of the first bone and second bone around the lengthwise axis of the body.

In one form, the implant body has a first diameter over a first length portion of the body and a second diameter, different than the first diameter, over a second length portion of the body.

In one form, the first length portion of the body has a first axis portion, the second length portion of the body has a second axis portion, and the first and second axes portions are coincident.

In one form, the first length portion of the body has a first axis portion, the second length portion of the body has a second axis portion, and the first and second axes portions are non-coincident.

In one form, the first length portion of the body has a first axis portion, the second length portion of the body has a second axis portion, and the first and second axes portions are non-parallel.

In one form, the implant is made from at least one of: a) allograft bone; b) metal; c) polyetheretherketone (PEEK); d) bioabsorbable material; e) calcium salt; f) titanium; g) stainless steel; h) carbon fiber; and i) polyetherketoneketone (PEKK).

In one form, the method of fusing first and second bones at a joint further comprises the step of separating the suture from the implant after exerting the force through the component.

In one form, the implant is a first implant. The method of fusing first and second bones at a joint further includes the steps of obtaining a second implant and engaging the second implant with the first and second bones.

In one form, the step of engaging the second implant involves engaging the second implant with the first and second bones so that parts of the first and second bones are urged towards each other.

In one form, the second implant is made up of a suture.

In one form, the step of engaging the second implant with the first and second bones involves forming the suture into a loop that acts against parts of the first and second bones.

In one form, the loop is a continuous loop.

In one form, the loop is in the form of a box.

In one form, the loop is in the form of a figure eight.

In one form, the loop is in the form of both a figure eight and a box.

In one form, the method of fusing first and second bones at a joint further includes the steps of obtaining a sound and directing a part of the sound into the formed receptacle on at least one of the first and second bones to check for at least one of: a) a desired depth; b) desired shape; and c) desired effective diameter of the formed receptacle.

In one form, the invention is directed to a kit usable for fusing first and second bones at a joint formed between a first end of the first bone and a first end of the second bone. The kit includes: an implant having a body and a length between first and second ends; at least one tool configured to form: a) a first receptacle by modifying an intramedullary space of the first bone; and b) a second receptacle by modifying an intramedullary space of the second bone; and a drawing component configured to releasably engage and exert a force on the implant body with the first end of the first bone against or adjacent to the first end of the second bone and a length portion of the implant body extended into the first receptacle on the first bone so as to thereby: a) move a part of the length portion of the implant body out of the first receptacle; and b) move one of the first and second ends of the implant body one of: i) into the second receptacle; and ii) further within the second receptacle.

In one form, the drawing component is a suture. The implant has an opening through which the suture can be directed to allow the suture to be doubled against itself to define a bight portion that can be drawn against the implant body.

In one form, the kit is provided in combination with a suture engaging tool that has a part configured to be directed through the second receptacle in a first direction to engage the suture and thereafter be moved oppositely to the first direction to be withdrawn from the second receptacle with the engaged suture to thereby expose a graspable part of the suture.

In one form, the kit is provided in combination with a suture engaging tool that has a part configured to be directed through the second receptacle in a first direction out the second end of the second bone and engage the suture and thereafter moved further in the first direction to be withdrawn from the second receptacle with the engaged suture to thereby expose a graspable part of the suture.

In one form, the drawing component has a connector that can be releasably engaged with a connector on the implant.

In one form, the connectors on the drawing component and implant are releasably threadably engaged.

In one form, the opening in the implant is at a location between the first and second ends of the implant.

In one form, the implant body has a non-uniform diameter between the first and second ends of the implant.

In one form, the implant has a body with a lengthwise axis and an external surface that extends around the lengthwise axis. The external surface has a non-circular shape as viewed along the lengthwise axis over at least a portion of the length of the implant. With the first bone and second bone in a fused relationship, the non-circular shape of the external surface causes the implant to be keyed against turning relative to at least one of the first bone and second bones around the lengthwise axis of the body.

In one form, the implant body has a first diameter over a first length portion of the implant body and a second diameter, different than the first diameter, over a second length portion of the implant body.

In one form, a first length portion of the implant body has a first axis portion, a second length portion of the implant body has a second axis portion, and the first and second axes portions are coincident.

In one form, a first length portion of the implant body has a first axis portion, a second length portion of the implant body has a second axis portion, and the first and second axes portions are non-coincident.

In one form, a first length portion of the implant body has a first axis portion, a second length portion of the implant body has a second axis portion, and the first and second axes portions are non-parallel.

In one form, the implant is made from at least one of: a) allograft bone; b) metal; c) polyetheretherketone (PEEK); d) bioabsorbable material; e) calcium salt; f) titanium; g) stainless steel; h) polyetherketoneketone (PEKK), and i) carbon fiber.

In one form, the kit usable for fusing first and second bones at a joint is provided in combination with at least one sound having a projecting length configured to be extended into at least one of the first and second receptacles to thereby predetermine a relationship between the at least one of the first and second receptacles and a part or parts of the implant that is/are directed into the at least one of the first and second receptacles.

In one form, the implant is a first implant. The kit is provided in further combination with a second implant made up of a suture.

In one form, the suture is an osseous suture.

In one form, the drawing component is a suture. The kit is provided in further combination with a suture engaging tool that has a suture engaging part that is configured to engage the suture and be advanced through the second receptacle so that a part of the suture is advanced through and out of the second receptacle.

In one form, the suture engaging tool includes a cutting tool to modify the intramedullary space of at least one of the first and second bones to allow the cutting tool to be advanced fully through the at least one of the first and second bones.

In one form, the suture engaging tool has a body. The suture engaging part and cutting tool are on the body of the suture engaging tool.

In one form, the body of the suture engaging tool has a length between spaced ends. The suture engaging part and cutting tool are spaced from each other lengthwise of the body of the suture engaging tool.

In one form, the suture engaging part is adjacent one of the spaced ends of the body of the suture engaging tool. The cutting tool is adjacent the other of the spaced ends of the body of the suture engaging tool.

### BRIEF DESCRIPTION OF THE DRAWINGS

FIG. 1 is a plan view of one exemplary joint location between two bones at which the present invention can be practiced;

FIGS. 2-6 show sequentially a prior art technique for fusing the bones in FIG. 1 using a K-wire/pin;

FIG. 7 is a view corresponding to that in FIG. 3 and showing another conventional technique for fusing the same bones with one or more K-wires/pins implanted in a different manner;

FIG. 8 is a view as in FIG. 7 showing another conventional technique wherein threaded fasteners are used instead of the K-wires/pins in FIG. 7;

FIG. 9 is a schematic representation of another conventional technique for fusing bones and utilizing an external implant/plate;

FIG. 10 is a fragmentary, partially schematic representation showing another form of prior art implant that penetrates intramedullary spaces of fused bones;

FIG. 11 is a flow diagram representation of a method of fusing first and second bones at a joint, according to the invention;

FIG. 12 is a schematic representation of a kit usable for fusing first and second bones at a joint, according to the invention;

FIG. 13 is a side view of the bones in FIG. 1;

FIG. 14 is a view as in FIG. 13 wherein cooperating surfaces on the bones have been prepared/modified to define complementary confronting surfaces;

FIG. 15 is a plan view of the components in their FIG. 14 state;

FIG. 16 is a view as in FIG. 15 wherein receptacles/bores have been formed in the bones;

FIG. 17 is a side view of the bones in their FIG. 16 state;

FIG. 18 is a plan view of a first of the bones in FIG. 16, with one form of the inventive implant directed into the receptacle/bore formed on the first bone;

FIG. 19 is a side view of the components in the FIG. 18 state;

FIG. 20 is a view as in FIG. 18 wherein the implant has been directed further into the first bone;

FIG. 21 is a side view of the components in the state in FIG. 20;

FIG. 22 is a view as in FIG. 21 wherein a suture engaging tool has been directed through the second bone in preparation to engage a suture/drawing component attached to the implant;

FIG. 23 is a view as in FIG. 22 wherein the suture has been engaged with the suture engaging tool;

FIG. 24 is a view as in FIG. 23 wherein the suture engaging tool has been repositioned to draw the suture through and out of the second bone;

FIG. 25 is a top view of the components as in FIG. 24 and with the first and second bones placed in a fused relationship;

FIG. 26 is a side view of the components in the state in FIG. 25;

FIG. 27 is a view as in FIG. 26 wherein the suture has been pulled to exert a force on the implant that moves the implant further into the second bone;

FIG. 28 is a side view of the components in the FIG. 27 state;

FIG. 29 is a view as in FIG. 27 wherein the suture has been separated from the implant and removed;

FIG. 30 is a side view of the components in the state of FIG. 29;

FIG. 31 is a view as in FIG. 21 and using a different form of implant, according to the invention, and a drawing component, according to the invention, that is different than the suture in the earlier embodiment;

FIG. 32 is a view as in FIG. 31 wherein the bones have been placed into an aligned lengthwise relationship and with the drawing component in FIG. 31 releasably engaged with the implant;

FIG. 33 is a view as in FIG. 32 wherein the bones are moved against each other into a fused relationship;

FIG. 34 is a view as in FIG. 33 wherein the drawing component has been manipulated to move the implant from the FIG. 33 position further into the second bone;

FIG. 35 is a view as in FIG. 34 wherein the drawing component has been disengaged from the implant and separated;

FIGS. 36 and 37 are schematic representations showing further forms of implants, according to the invention, with different relationships between non-colinear length portions;

FIG. 38 is a fragmentary, schematic depiction of a tapered length of an implant, according to the invention;

FIGS. 39 and 40 show different cross-sectional shapes of outer implant surfaces, according to the invention;

FIG. 41 is a schematic representation of an implant body with circumferentially extending and axially spaced projections, according to the invention;

FIG. 42 is a schematic representation of an implant body, according to the invention, with an external surface having longitudinally/axially extending ribs thereon;

FIG. 43 is a flow diagram representation of a modified form of method, according to the present invention, for fusing first and second bones at a joint;

FIG. 44 is a side view of first and second bones, as in FIG. 17, and with a bore formed through each of the first and second bones;

FIG. 45 is a top view of the bones as in FIG. 44 wherein a suture/implant has been threaded through the bores, as shown in FIG. 44, in a figure eight pattern between the first and second bones;

FIG. 46 is a side view of the components in the FIG. 45 state;

FIG. 47 is a top view of a modified form of implant, and with the implant engaged with a drawing component/suture, as shown in FIGS. 18 and 19;

FIG. 48 is a side view of the components in the FIG. 47 state;

FIG. 49 is a view corresponding to that in FIG. 22 and with a modified form of suture engaging tool;

FIG. 50 is a view of the components in FIG. 49 wherein the suture engaging tool is engaged with the drawing component/suture;

FIG. 51 is an enlarged, fragmentary view showing a suture engaging part on the suture engaging tool of FIG. 50 and having a slot formed thereon and with suture lengths placed therein;

FIG. 52 is a view as in FIG. 51 wherein a body of the suture engaging tool has been crimped to fix the suture lengths to the body of the suture engaging tool;

FIG. 53 is a view as in FIG. 50 wherein the suture engaging tool has been directed through and from the second bone to define a grasping portion/length for the suture;

FIG. 54 is a top view of the components in FIG. 53 wherein the surfaces on the bones have been brought into apposed relationship;

FIG. 55 is a side view of the components in the FIG. 54 state;

FIG. 56 is a view as in FIG. 54 wherein the drawing component/suture has been pulled to reposition the implant from the position in FIGS. 54 and 55;

FIG. 57 is a side view of the components in the FIG. 56 state;

FIG. 58 is a view as in FIG. 56 wherein the drawing component/suture has been removed;

FIG. 59 is a side view of the components in the FIG. 58 state;

FIG. 60 is a view as in FIG. 58 wherein the suture/implant has been tightened to urge surfaces on the first and second bones against each other;

FIG. 61 is a side view of the components in the FIG. 60 state;

FIG. 62 is a schematic representation of the inventive suture/implant cooperating between first and second bones;

FIG. 63 is a schematic representation of another form of implant, according to the present invention;

FIG. 64 is a schematic representation of anchors usable to cooperate with a suture/implant, according to the invention;

FIG. 65 is a side elevation view of a sound, according to the invention, usable to check the configuration of a receptacle for the implant and with one form of projecting length;

FIG. 66 is a fragmentary elevation view of a modified form of a projecting length on a sound, as in FIG. 65, according to the invention; and

FIG. 67 is a view as in FIG. 44 and showing a countersunk bore/receptacle being formed in one of the bones.

### DETAILED DESCRIPTION OF THE PREFERRED EMBODIMENT

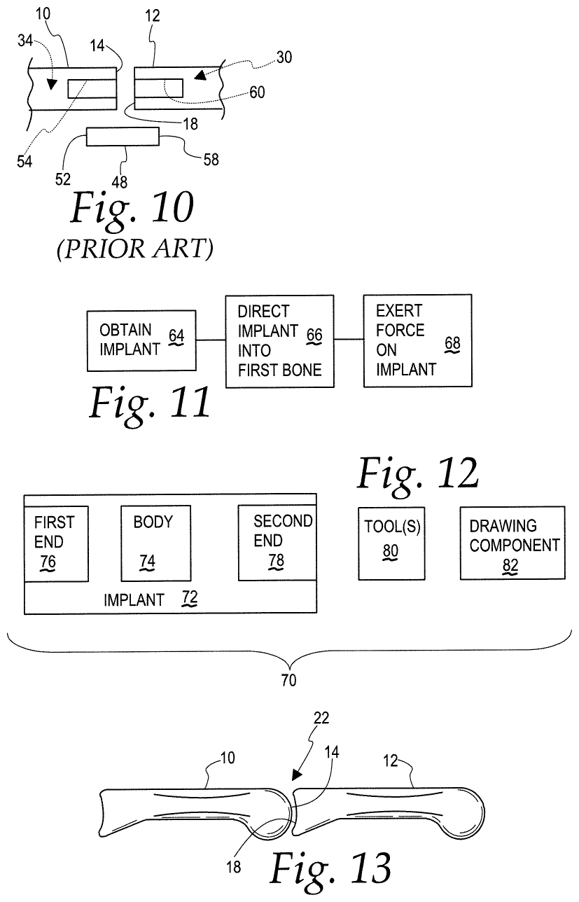

In FIG. 11, a method of fusing first and second bones at a joint, according to the invention, is shown in schematic form. The second bone has first and second ends. A first end of the first bone is at the first end of the second bone where the joint is formed.

As shown at block 64, an implant is obtained having a length between first and second ends.

As shown at block 66, the implant is directed into an intramedullary space of the first bone so that a first length portion of the implant resides within the intramedullary space of the first bone.

As shown at block 68, a force is exerted on the implant through an opening at the second end of the second bone, as an incident of which: a) a part of the implant within the intramedullary space of the first bone moves out of the intramedullary space of the first bone; and b) one of the first and second ends of the implant is caused to move one of: i) into the intramedullary space of the second bone; and ii) further within the intramedullary space of the second bone towards the second end of the second bone.

In one form, the method of FIG. 11 is performed using a kit, as shown schematically at 70 in FIG. 12. The kit consists of an implant 72 with a body 74 having a length between first and second ends 76, 78 of the implant 72.

At least one tool 80 is configured to form at least one of: a) a first receptacle/bore by modifying an intramedullary space of the first bone; and b) a second receptacle/bore by modifying an intramedullary space of the second bone.

A drawing component 82 is configured to releasably engage and exert a force on the implant body 74 with the first end of the first bone against or adjacent to the first end of the second bone and a length portion of the implant body extended into the first receptacle on the first bone so as to thereby: a) move a part of the length portion of the implant body out of the first receptacle; and b) move one of the first and second ends of the implant body one of: i) into the second receptacle; and ii) further within the second receptacle.

The schematic depictions of the method in FIG. 11 and the kit in FIG. 12 are intended to encompass virtually an unlimited number of variations of depicted structures/components and how they are used.

For example, the method in FIG. 11 is intended to encompass virtually any method wherein with the implant length portion residing within the first bone, the first and second bones can be aligned in, or approximately in, a final fused relationship whereupon the implant is moved so that at least part of the length of the implant within the first bone at the first end of the first bone can be shifted so that an end of the implant 72 can be advanced into, or further into, the second bone without requiring excessive distraction between the first and second bones that might result in tissue damage. That is, the implant 72 can be initially embedded so that no part projects from the first end of the first bone as the second bone is aligned with the first bone to establish the desired fused relationship between the first and second bones. Alternatively, a degree of projection may be such that little or no excessive distraction between the bones is required to align the projecting end of the implant with its receptacle in the second bone.

The schematic depiction of the implant in FIG. 12 is intended to encompass implant configurations that vary significantly, with only exemplary forms described in further detail below.

Likewise, the drawing component 82 may take an unlimited number of forms which permit a free end of the implant, with the implant initially embedded in the first bone, to be directed into the second bone and moved therewithin without requiring any, or excessive, distraction of the second bone. The nature of the drawing component 82 may vary considerably, with only exemplary forms described hereinbelow.

Specific exemplary forms of the inventive method and kit will be described hereinbelow, with it being understood that these are exemplary in nature only and should not be viewed as limiting.

In many of the drawings, the first and second bones are shown in different relationships with substantial spacing and lengthwise misalignment—corresponding to substantial distraction of one bone relative to the other. These exaggerated relationships are made only to allow clear explanation of the invention.

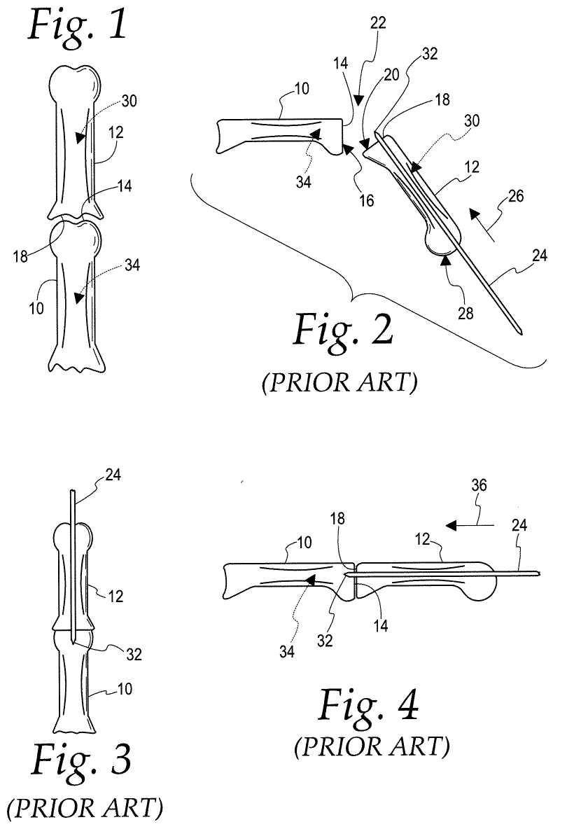

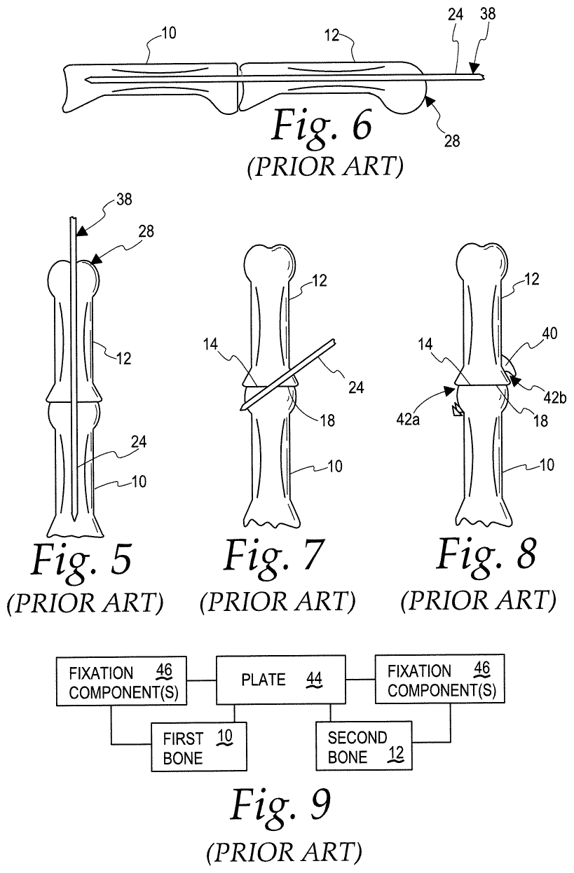

The description of the invention with respect to the joint between the proximal phalanx 10, as shown in FIG. 1, and the middle phalanx 12, is but exemplary of the many joints/sites at which the invention can be practiced.

While FIG. 1 depicts a joint between a proximal phalanx 10 and a middle phalanx 12, it should be understood that the two bones could be, for example, a middle and distal phalanx of a finger or toe or an interphalangeal joint of thumb or great toe or other bones at a joint.

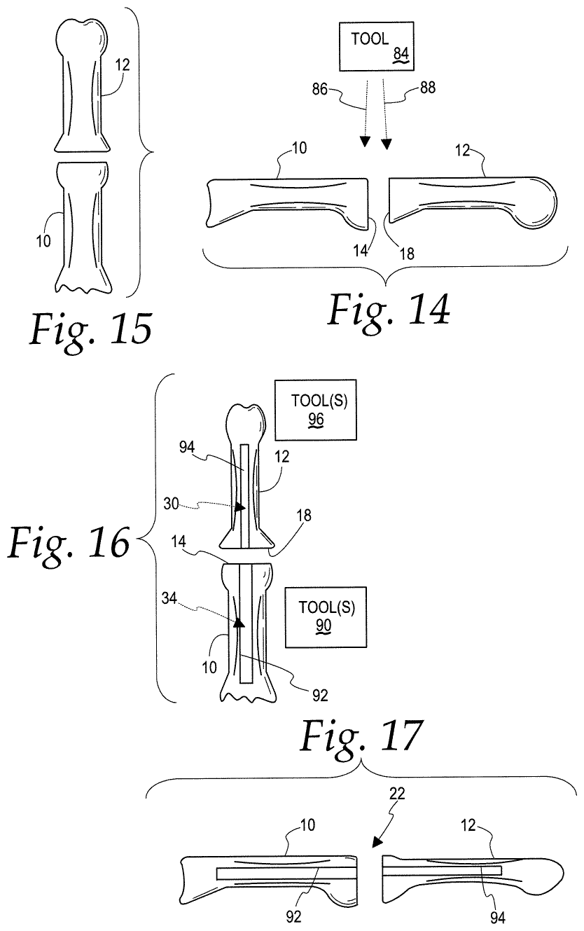

FIGS. 1 and 13 show the natural configuration of the surfaces 14, 18, respectively on the first and second bones 10, 12 at the representative joint location 22.

While not required, in a first step, as shown in FIG. 14, the surfaces 14, 18 are preferably prepared/modified, as by one or more tools 84 advanced in the direction of the arrows 86, 88 to respectively cause the surfaces 14, 18 to each have a flat shape, so that with the planes of the flat shapes apposed, the confronting surfaces 14, 18 will cause the first and second bones 10, 12 to be maintained in a desired lengthwise alignment. In the depicted example, the alignment of the bones 10, 12 is such that their lengths are substantially colinear. However, as described in greater detail below, the surfaces 14, 18 can be prepared so that with the first and second bones in the fused relationship the first bone is in an angular relationship with the second bone 12—such as in mild flexion.

The surface 14, 18 may be modified to have any shapes, other than flat, that cooperate to facilitate maintaining of a desired fused relationship and establish adequate fusing area between the bones, as mentioned in the Background Art section above. Moreover, the tool(s) 84 used to prepare the surfaces may be advanced in a direction that is colinear with the long axis of either the first bone 10 or the second bone 12.

As shown in FIGS. 16 and 17, at least one tool 90 is used to form a receptacle/bore 92 in the first bone 10 which enlarges/modifies the intramedullary space 34 of the first bone 10.

The same or a similar tool(s) 96 is used to produce a receptacle/bore 94 in the second bone 12 that enlarges/modifies the intramedullary space 30 of the second bone 12.

For purposes of simplicity, the receptacles/bores 92, 94 will be considered to be parts of their respective intramedullary spaces 30, 34.

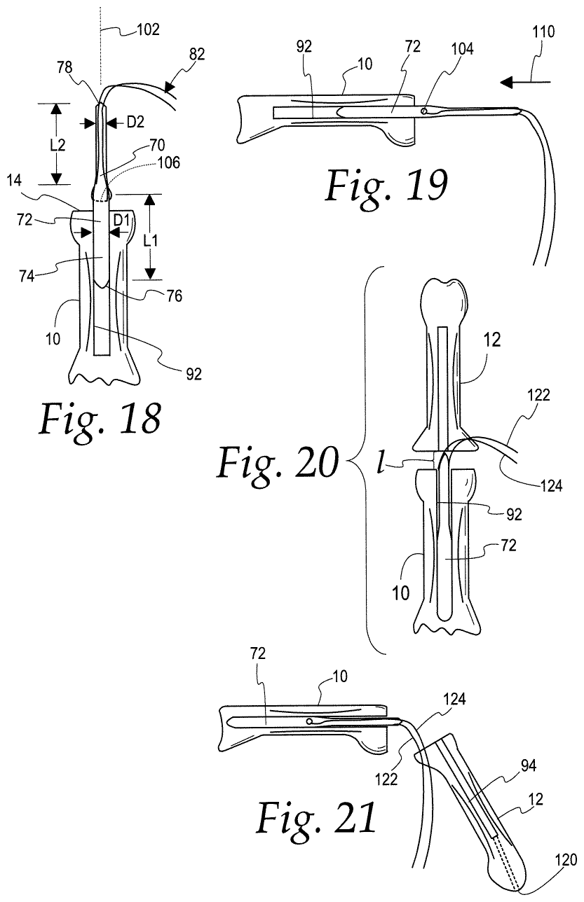

As shown in FIGS. 18-30, a first form of implant 72 is used which, as noted above, is but exemplary of the many different forms of the implant contemplated within the schematic showing in FIG. 12. The implant 72 has an elongate body 74 between a first end 76 and a second end 78 of the implant 72.

In this embodiment, the body 74 of the implant 72 has a non-uniform diameter between the ends 76, 78 of the implant 72.

More specifically, the implant body 74 has a first length portion L1 with an effective diameter D1 that is greater than the effective diameter D2 of a second length portion L2 of the implant body 74. The diameter is characterized as “effective” since the invention contemplates within the schematic depiction of the body 74, that it may have, as viewed along the lengthwise axis 102 of the body 74, a circular or non-circular shape and/or surface variations, such as discrete projections, recesses, etc.

For example, the body 74 may have surface irregularities that facilitate frictional gripping within the bones 10, 12, with exemplary shapes being longitudinal ribs, circumferential ribs, teeth, threads, etc., as described in further detail below. The shapes and surface contours may extend over the entire length of the body 74, over a portion of the length thereof, be located at discrete locations thereon, etc. Again, the schematic depiction of the body 74 is intended to encompass these and other shapes that a person skilled in the art would readily arrive at, based upon the teachings herein, to enhance a holding force, as hereinafter described.

In the depicted form, the length portion L1 of the body 74 is designed to be press fit and snugly held within the receptacle/bore 92 on the first bone 10. The end 76 is rounded to align and smoothly guide the implant 72 into place.

Similarly, the length portion L2 is configured to be slidable into the receptacle/bore 94 and snugly maintained therewithin with the end 78 thereof similarly rounded.

The effective diameters of the receptacles/bores 92, 94 are different to match their respective engaging parts on the implant body 74. It should be noted that the body 74 might have a uniform effective diameter which might dictate making the receptacles/bores 92, 94 of like diameter.

In one form, the drawing component 82, depicted schematically in FIG. 12, is in the form of a suture. “Suture” is intended to encompass a conventional suture material, a flexible wire, a cable, etc.

An end of the suture 82 is directed through an opening 104 in the implant 72. The suture 82 is then doubled back on itself to produce a bight portion 106 that bears against the implant as a force is exerted through the suture as it is drawn in the direction of the arrow 108 in FIG. 24.

As depicted, the opening 104 is at a location approximately midway between the ends 76, 78 of the implant 72. In other forms, the opening 104 may be closer to the end 76 or the end 78.

As seen in FIG. 18, the end 76 of the implant 72 is initially directed into the receptacle/bore 92 of the first bone 10, passing through the surface 14. This draws a portion of the suture 82 to within the receptacle/bore 92.

As shown in FIG. 19, the implant 72 is thereafter advanced in the direction of the arrow 110 further into the receptacle/bore 92 to the position as shown in FIGS. 20-22, wherein only a short length/protrudes from the receptacle/bore 92.

A suture engaging tool 112 may be part of the kit 70, as shown schematically in FIG. 12. In the embodiment shown, the suture engaging tool 112 has an elongate, rod-like body 114 with a suture engaging part 116 at one end and a cutting tool 118 at the opposite end. While not required, in this particular configuration, the single suture engaging tool 112 performs two functions.

In one alternative form, the suture engaging tool 112 may have the cutting tool 118 on the same end as the suture engaging part 116, allowing the suture engaging tool to be initially advanced: a) from the second end 28 into the intramedullary space 30 and out the first end 20 of the second bone 12; or b) from the first end 20 into the intramedullary space 30 and out the second end 28.

In another alternative form, the suture engaging tool consists of two separate components—one in the form of a cutting tool, to function in preparation of at least the receptacle/bore 94, and a second to engage the suture 82 so as to facilitate repositioning thereof.

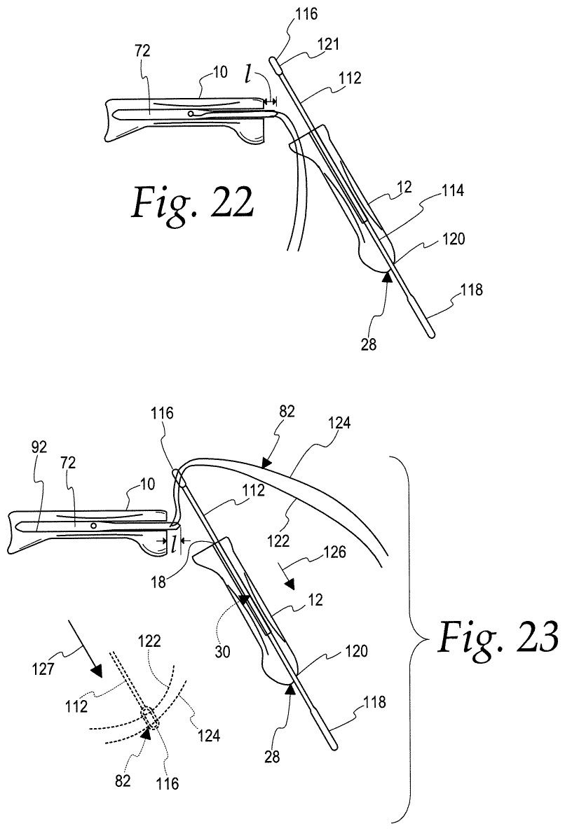

Initially, the cutting tool 118 is used to define, or enlarge, an opening 120 at the second end 28 of the second bone 12 through which the end of the body 114 with the suture engaging part 116 can be directed. The suture engaging tool 112 may be used to form/enlarge the opening 120 by advancing the suture engaging tool 112 with the cutting tool 118 in either a leading or trailing direction. The opening 120 is in communication with the receptacle/bore 94 so that the body 114 can be inserted into and advanced fully through the second bone 12, to a position as shown in FIG. 22.

In the configuration shown, the suture engaging part 116 has an exposed closed loop 121 through which doubled back lengths 122, 124 of the suture can be directed, as shown in FIG. 23.

By moving the suture engaging tool 112 in the direction of the arrow 126 in FIG. 23-in one form oppositely to the insertion direction-the suture engaging part 116 will draw the suture lengths 122, 124 out of the opening 120 at the second bone end 28, thereby exposing a grasping portion/length 12 on each of the aforementioned suture lengths 122, 124.

Alternatively, as shown in dotted lines in FIG. 23, the suture engaging tool 112 might engage the suture 82, in the same way as shown in solid lines, whereupon the suture engaging part 116 is directed through an opening at the surface 18 at the first end of the second bone 12 into and through the intramedullary space 30 of the second bone 12 to out the opening 120, thereby exposing the graspable length 12.

With the components in the FIG. 24 arrangement, the first and second bones 10, 12 can be relatively moved to cause the protruding end 78 of the implant 72 to be advanced through the surface 18 and into the receptacle/bore 94 on the second bone 12. The protruding length l of the implant 72, when directed into the receptacle/bore 94, is preferably adequate to establish, and at least preliminarily maintain, the predetermined, desired fused relationship between the first bone 10 and the second bone 12.

In the depicted form, the implant 72 has a straight central axis 102 between the ends 76, 78, whereby the axes of the receptacles/bores 92, 94 substantially coaxially align to be coincident with the bones 10, 12 in the fused relationship.

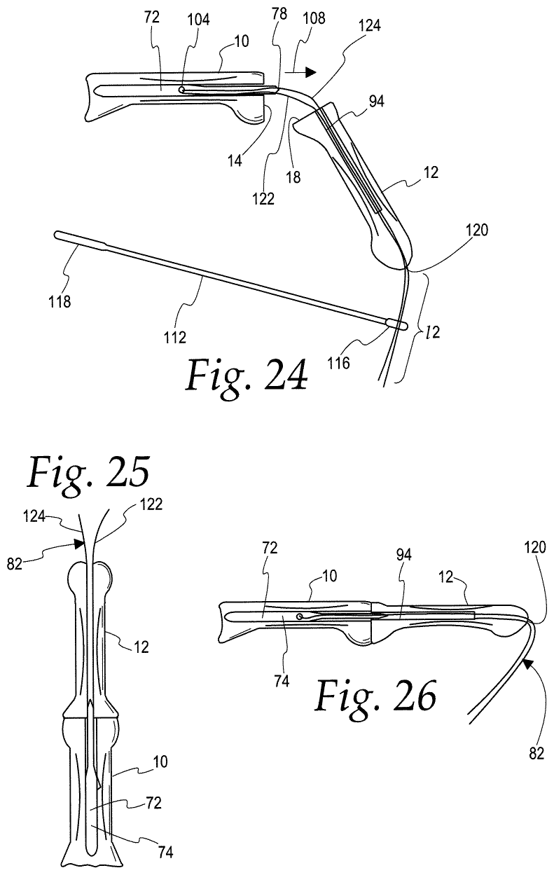

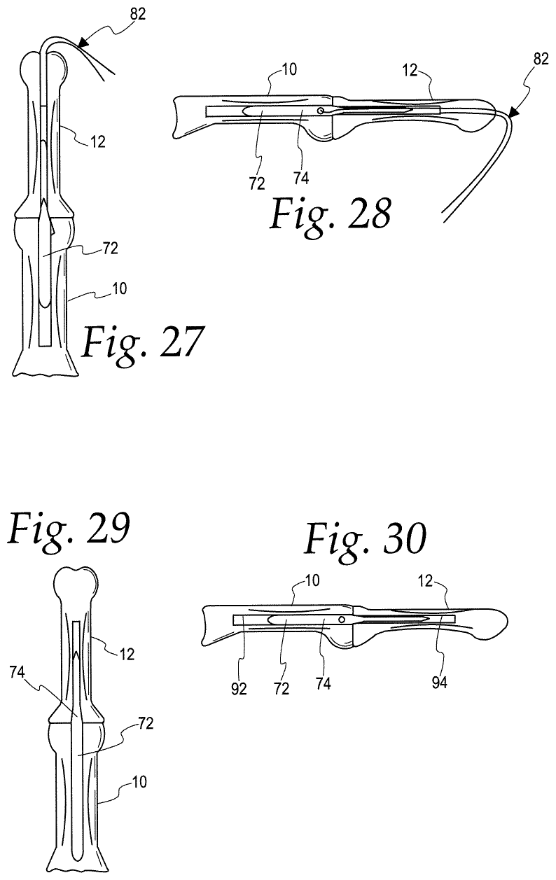

Once the fused relationship is preliminarily at least approximately established, a force is exerted on the implant 72, in the direction of the arrow 108, by drawing on the grasping length 12 of the suture 82. This action changes the implant 72 from the position shown in FIGS. 25 and 26 to that shown in FIGS. 27-30, wherein a significant portion of the length initially embedded in the receptacle/bore 92 shifts into the receptacle/bore 94. As a result, the implant achieves extended load bearing lever arms within the intramedullary spaces of each bone 10, 12 that effectively resists angular movement-something that may not be practically achievable with the prior art as shown in FIG. 10.

The implant 72 is designed, and the receptacles/bores 92, 94 configured, so that in one exemplary starting state, as shown in FIG. 20, a relationship can be established wherein most, or potentially all, of the implant 72 resides within the receptacle/bore 92 and further wherein in a final position of the implant 72, as shown in FIG. 27, there is adequate penetration of both the receptacles/bores 92, 94 by the implant 72 that the bones 10, 12 are adequately stabilized and maintained against relative movement.

As shown in FIGS. 29 and 30, the suture 82 can be removed at the end of the procedure, though this is not always a requirement.

It is possible to construct the implant 72 so that there is no projection from the first bone 10, when in a state corresponding to the FIG. 20 state, wherein the second bone 12 does not have to be distracted significantly, by movement away from the first bone end, to align the implant end 78 with the receptacle/bore 94. Thus, when the suture 82 is drawn, starting from a state corresponding to that in FIG. 25, the implant end 76 is caused to move both into and along the receptacle/bore 94.

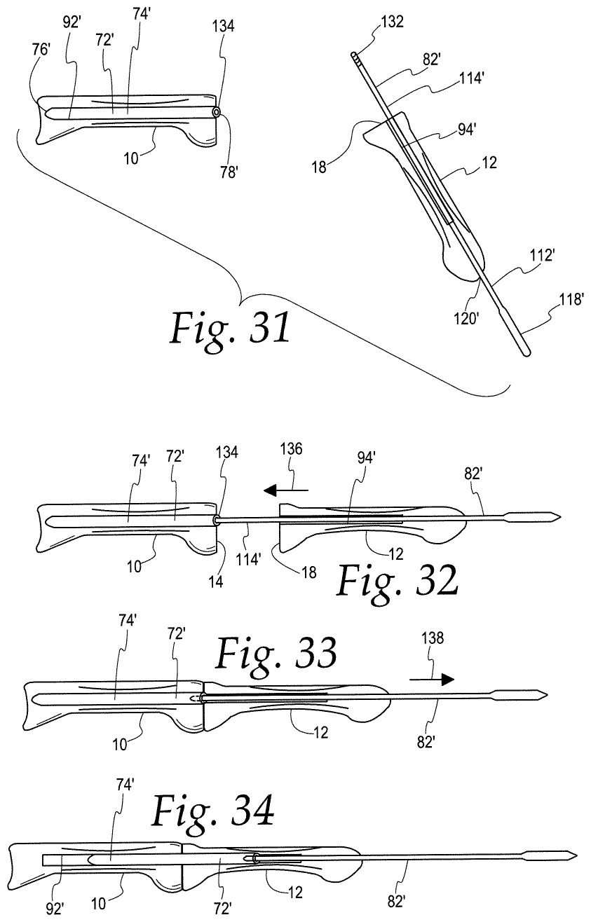

In FIGS. 31-35, a modified form of implant 72′ is shown.

The implant 72′ has a body 74′ with a substantially uniform diameter between ends 76′, 78′ of the implant 72′—which are both rounded to prevent hangup. That is, the body 74′ has a straight lengthwise axis between the ends 76′, 78′ of the implant 72′. In alternative forms this diameter is non-uniform.

In most respects, the method performed using the implant 72′ is substantially the same as the method performed using the implant 72.

The corresponding receptacles/bores 92′, 94′, in this exemplary form, have the same diameter to cooperate with the body 74′.

The implant 72′ is directed into the bone 10, with the FIG. 31 position for the implant 72′ corresponding generally to that for the implant 72 shown in FIG. 22.

Rather than using a suture as the drawing component 82, a drawing component 82′ is integral with a component 112′ corresponding to the suture engaging tool 112 and is designed to releasably mechanically connect to the implant end 78′.

The drawing component 82′/component 112′ incorporates features of the suture engaging tool 112. The component 82′/component 112′ has an elongate rod-like body 114′ with a cutting tool 118′ at one end thereof. At the opposite end thereof, a connector 132 is provided to cooperate with a connector 134 at the end 78′ of the implant 72′. This multi-function design of the drawing component 82′/component 112′ is a convenience but not a requirement, as explained for the drawing component 82, above.

The drawing component 82′/component 112′ can be used to form/enlarge the opening 120′, with the drawing component 82′/component 112′ either as in FIGS. 31 and 32 or inverted from that orientation.

In one form, before the FIG. 31 relationship between the second bone 12 and drawing component 82′/component 112′ is established, the drawing component 82′/component 112′ is inverted from the FIG. 31 position and the cutting tool 118′ utilized to form, or enlarge, the opening 120′, corresponding to the opening 120, as shown in FIGS. 21 and 22. Once the opening 120′ is formed, the drawing component 82′ can be directed, leading with the connector 132, through the opening 120′ and into and through the bore 94′ to be exposed beyond the surface 18 of the second bone 12.

With the bone parts 10, 12 generally in the FIG. 32 relationship-in lengthwise alignment—the second bone 12 can be advanced in the direction of the arrow 136, relative to the first bone 10, to place the surfaces 14, 18 in apposed relationship for fusion. The body 114′ aligns and guides movement of the second bone 12 towards the first bone 10 as this occurs.

It should be noted that the surfaces 14, 18 can be brought into confronting relationship before the drawing component 82′/component 112′ is directed through the second bone 12. In that case, a slight projecting length at the connector 134 may register with the receptacle/bore 94′ to at least approximately establish the desired fused relationship between the first bone 10 and the second bone 12.

As depicted, the connector 132 is a male threaded part that cooperates with a female threaded receptacle making up the connector 134. Of course, this male/female relationship might be reversed and other cooperating connector configurations are contemplated.

With the first bone 10 and second bone 12 at least generally in the fused relationship of FIG. 33, the drawing component 82′/component 112′ can be pulled in the direction of the arrow 138, which causes the implant 74′ to shift from the FIG. 33 position into the FIG. 34 position, whereupon an additional length thereof penetrates the second bone 12 to stably connect with the second bone 12.

Once the FIG. 34 state is realized, the drawing component 82′/component 112′ can be turned to disengage the connectors 132, 134, thereby allowing separation of the drawing component 82′/component 112′ from the bones 10, 12, as shown in FIG. 35.

As noted previously, the implants 72 are designed, and the cooperating bones 10, 12 prepared, so that a predetermined length of the implant is frictionally held in each of the bones 10, 12.

The frictional engagement force can be controlled by controlling how the diameters of the receptacles/bores 92, 94 are matched to the effective diameters of the cooperating implant lengths.

In the depicted forms, the implants 72 have a single, central, longitudinal axis.

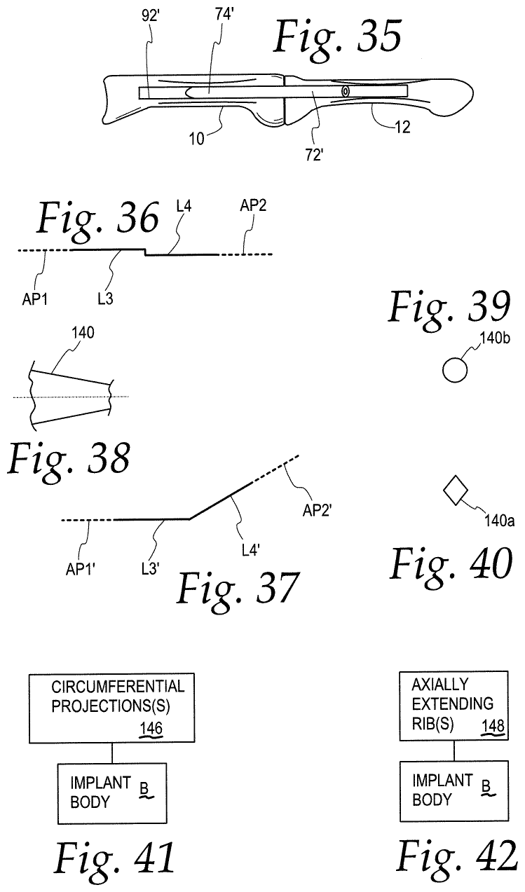

As shown schematically in FIG. 36, the body of an implant may have length portions L3, L4 that have axis portions AP1, AP2, respectively, that are parallel and offset.

In FIG. 37, corresponding length portions L3′, L4′ of an implant body have axis portions AP1′, AP2′ that are angled with respect to each other.

Other relationships between implant body length portions are contemplated.

As shown schematically in FIG. 38, an outer surface 140 over at least a portion of the length of an implant body may have a tapered diameter.

As shown in FIG. 39, the outer surface 140a of a particular implant body may be circular over its entire length or over only a portion thereof, as viewed along the implant axis.

In an alternative form, as shown in FIG. 40, the corresponding shape of the outer surface 140b may be non-circular over part or all of the implant length, with the depicted form exemplary in nature only.

As shown schematically in FIG. 41, at least one discrete circumferential projection 146 may be provided on an implant body B.

In FIG. 42, an axially extending rib, or axially extending and circumferentially spaced ribs 148 are shown schematically on an implant body B. A single rib 148 may also be provided.

All of these shapes are selected simply to identify representative shapes that can be incorporated to achieve the desired frictional holding force between the implant 72 and the bones 10, 12 while adapting to a particular bone size.

The implant can be made from many different materials or combinations thereof. As just examples, the implant may be made from one, or a combination of, allograft bone, metal, polyetheretherketone (PEEK), bioabsorbable material, calcium salt, titanium, stainless steel, polyetherketoneketone (PEKK), carbon fiber, etc.

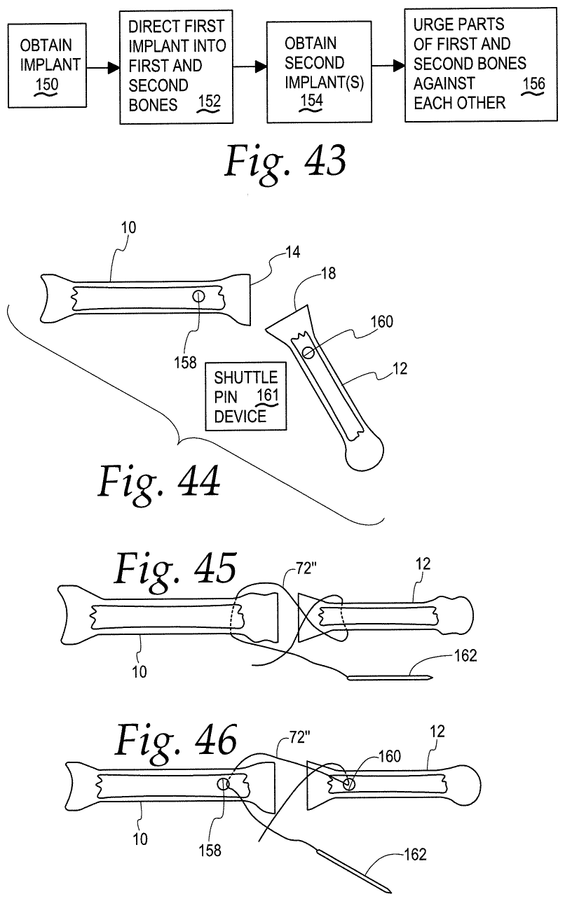

FIG. 43 is a flow diagram representation of a modified form of the inventive method of fusing first and second bones at a joint. As shown at block 150, an implant is obtained such as, but not limited to, the implants 72, described above and below.

As shown at block 152, the implant is directed into first and second bones, such as the first and second bones 10, 12, described above. As noted previously, the invention is not limited to the specific bones 10, 12, used as examples.

As shown at block 154, at least one supplemental implant is obtained.

As shown at block 156, at least one supplemental implant is utilized to urge parts of the first and second bones towards/against each other.

It is contemplated that the supplemental implant might be used in conjunction with the implants 72, 72′ described above, or a different type of implant. For purposes of explanation only, the supplemental implant will be described as used in conjunction with the exemplary implant 72″ hereinbelow with reference to FIGS. 44-61.

One exemplary form of the method, as shown in flow diagram form in FIG. 43, will now be described, also with reference to FIGS. 44-61.

In FIG. 44, the first and second bones 10, 12 are shown with the surfaces 14, 18, respectively thereon prepared to be placed in apposed relationship. Transverse bores 158, 160 are respectively formed in the first and second bones 10, 12, in the exemplary form approximately at the lengthwise location as indicated in FIG. 44. The bores 158, 160 may be formed by any suitable instrument, such as, but not limited to, the Assignee's “Shuttle Pin” device 161, which is the subject of U.S. Pat. No. 11,559,299, the disclosure of which is incorporated by reference. The holes 158, 160 are preferably at or central to the diaphyseal/metaphyseal junction.

As shown in FIGS. 45 and 46, the supplemental implant 72″ is in the form of a suture. Using a straight needle 162, or the Shuttle Pin device 161, the suture/implant 72″ is threaded through one of the bores 158, 160, crossed over the dorsal surface and threaded transversely through the other of the bores 158, 160 to create a “figure eight” pattern. As but one example, the suture/implant 72″ may be a #2-0 high molecular weight polyethylene suture material—which is an osseous suture material.

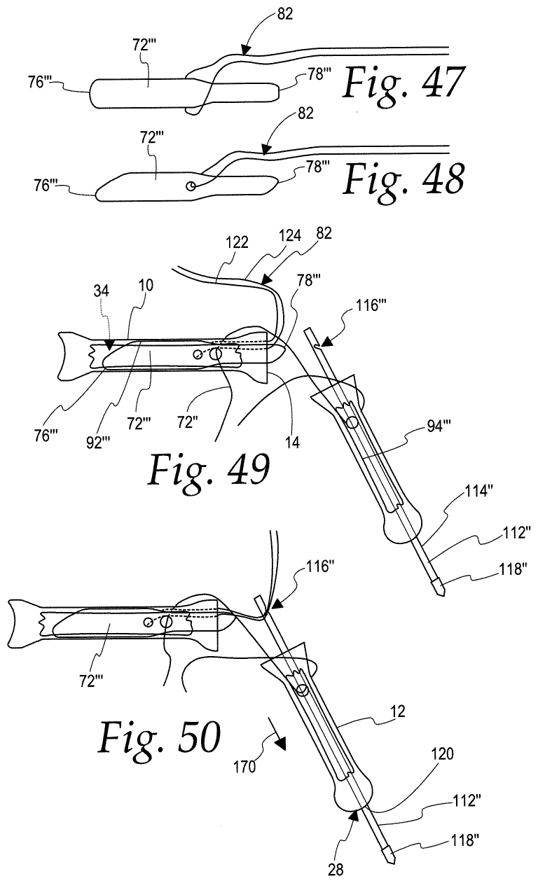

As shown in FIGS. 47 and 48, the aforementioned drawing component/suture 82 is directed through another modified form of the implant 72″ in the same manner as shown for the implant 72 in FIGS. 18 and 19. The drawing component/suture 82 may have the same construction as the suture/implant 72″, or a different construction.

As shown in FIG. 49, the implant 72″ is directed into the first bone 10, as the implant 72 is shown in FIG. 21, whereupon separate lengths of a U-shaped portion of the drawing component/suture 82 reside, one each, between the sides of the implant 72′' and the surface bounding the receptacle 92″. The suture lengths 122, 124 project outwardly from the intramedullary space 34 on the first bone 10 to beyond the surface 14. This step can be accomplished with the suture/implant 72″ remaining in a slackened state with a generally maintained wrapped configuration, as described above.

The depicted suture engaging tool 112″ may be the same as the aforementioned suture engaging tool 112, or the Shuttle Pin device 161, or may have another configuration. The suture engaging tool 112″/Shuttle Pin device 161 has a cutting tool 118″ at one end thereof and a suture engaging part 116″ spaced therefrom and, in this form, adjacent the opposite end of the body 114″ of the suture engaging tool 112″.

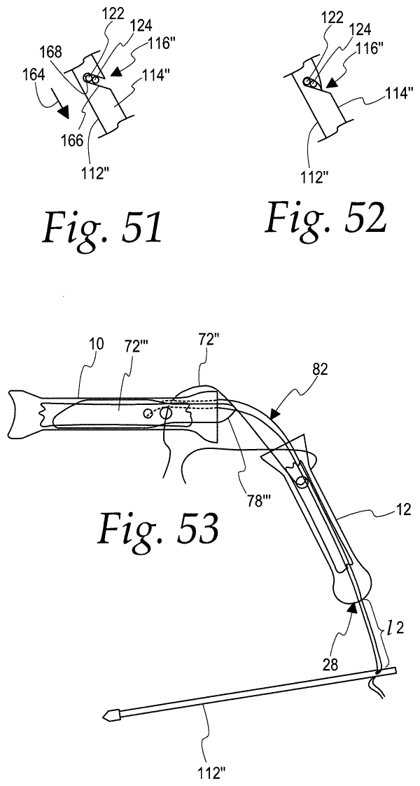

In the depicted embodiment, as seen clearly in FIG. 51, the suture engaging part 116″ has the general overall configuration disclosed in U.S. Pat. No. 11,559,299. With the body 114″ advanced in the direction of the arrow 164 in FIG. 51, the suture lengths 122, 124 are guided into an angled slot 166 whereupon the continued advancement of the body 114″ in the direction of the arrow 164 causes the surface 168 bounding the slot 166 to draw the suture lengths 122, 124 in following relationship.

Optionally, as shown in FIG. 52, the body 114″ can be crimped to collapse the slot 166, as further described in U.S. Pat. No. 11,559,299, whereupon the suture lengths 122, 124 are fixed with respect to the body 114″, which gives more positive control over movement of the drawing component/suture 82.

As shown sequentially in FIGS. 50 and 53, the suture engaging tool 112″ is advanced through the second bone 12 in the direction of the arrow 170, whereby the cutting tool 118″ produces or enlarges the aforementioned opening 120, thereby permitting the suture engaging tool 112″ to be advanced through the second bone 12 in the direction of the arrow 170 and to be separated therefrom as shown in FIG. 53, whereupon the grasping portion/length l2 projects from the end 28 of the second bone 12.

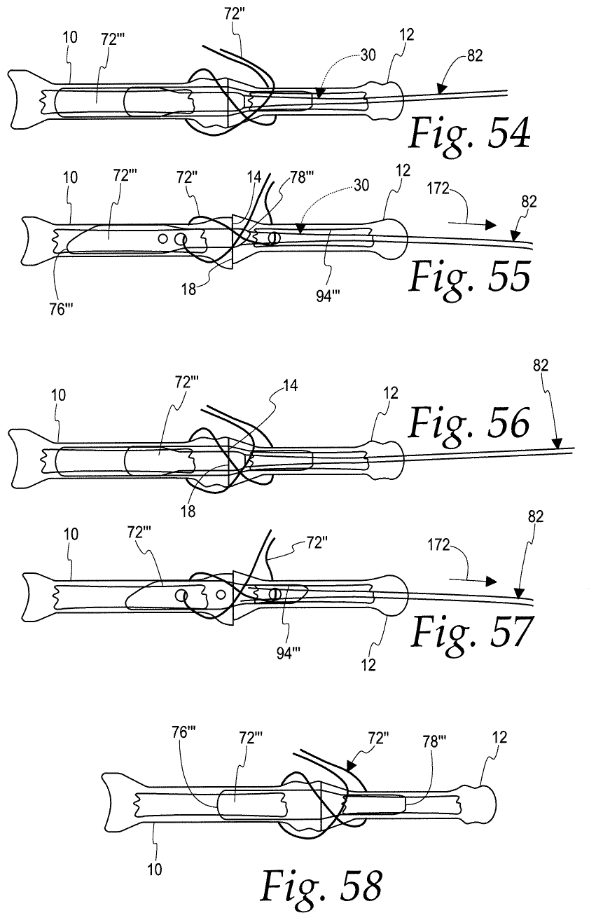

As shown in FIGS. 54 and 55, the projecting implant end 78″ can then be advanced into the intramedullary space 30 through the surface 18. The suture lengths 122, 124 reside, one each, between opposite sides of the implant 72″ and the surface bounding the receptacle 92″.

While maintaining the surfaces 14, 18 at least nominally in apposed relationship, the drawing component/suture 82 can be drawn in the direction of the arrow 172 in FIG. 55 to shift the implant 72″ from the position in FIGS. 54 and 55 to that in FIGS. 56 and 57.

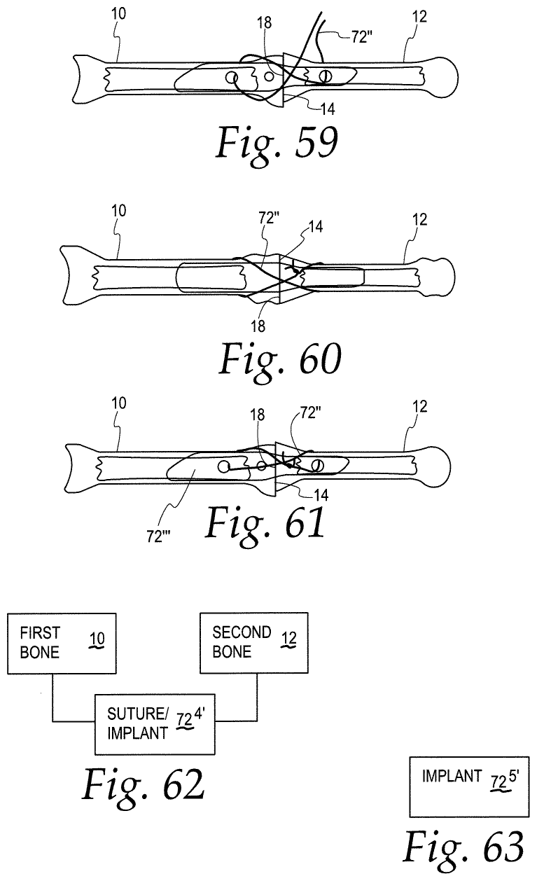

The drawing component/suture 82 can then be separated, as shown in FIGS. 58 and 59, whereupon the suture/implant 72″ can be tied in the depicted figure eight arrangement. While maintaining the bone surfaces 14, 18 compressed against each other, the suture/implant 72″ in the figure eight shape can be tightened and tied, as shown in FIGS. 60 and 61, which urges the bone surfaces 14, 18 towards/against each other.

Thereafter, the wound is closed and treated in conventional fashion.

It is contemplated that the suture/implant 72″ may be formed in different looped arrangements cooperating between and acting against the first and second bones 10, 12. FIG. 62 shows a suture/implant 72^{4′} that is intended to encompass virtually an unlimited number of different forming patterns for the suture/implant 72″ in relationship to the first and second bones 10, 12. For example, the arrangement may be a simple “box” shape or more complex routing of the suture/implant 72^{4′} acting between and against the first and second bones 10, 12. As one example, both a box and figure eight routing may exist at the same joint.

As shown in FIG. 63, it is also contemplated that yet another form of implant 72^{5′} might be utilized in conjunction with one or more of the implants 72, 72′, 72″, 72′″. The implant 72^{5′} may take any form of implant commonly used at joint surfaces including, but not limited to, an external implant that spans between the first and second bones 10, 12, respectively.

The suture/implant 72″, upon being tied and tightened, provides compression at the surfaces 14, 18, which promotes healing and creates additional stability between the first and second bones 10, 12.

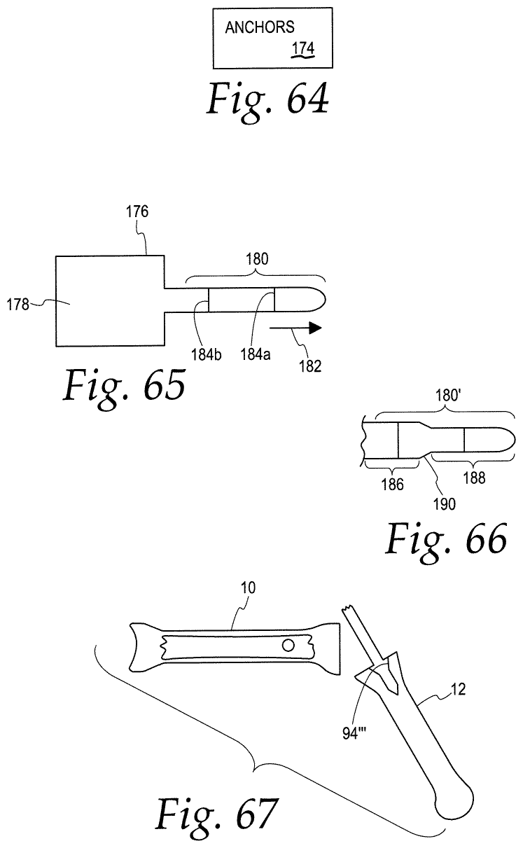

As shown in FIG. 64, as an alternative to producing bores through the bones 10, 12, one or more anchors 174 might be used to fix the suture/implant 72″ to one or both of the bones 10, 12 during the engagement process.

To assure that the receptacles/bores 92, 94 are configured appropriately for a particular implant, at least one sound 176, as shown in FIG. 65, is obtained and used as part of the aforementioned kit to preliminarily assess the relationship between the implants 72 and receptacles therefor to make certain the implant will be properly seated, be adequately held, produce desired multi-dimensional stability between bone parts, etc. The sound 176 has a graspable portion 178 and a penetrating length 180 projecting away from the graspable portion 178. The penetrating length 180 can be advanced in the direction of the arrow 182 into a bore/receptacle 92, 94 to be measured/inspected. The penetrating length 180 can be dimensioned to determine the frictional fit for the implant and has indicia 184a, 184b that are representative in nature only and give an indication of depth penetrated by the projecting length 180. The number of indicia and their precise location is dictated by a particular site and implant.

In FIG. 66, a modified projecting length 180′ is shown with length portions 186, 188 with different diameters and joined through a tapered transition region 190. This configuration may be used to test a countersunk bore 94′″, as shown in FIG. 67 which shows corresponding exemplary bones 10, 12.

The sound 176 may be made with many different configurations to be used with different implants, in each case to check for at least one of: a) desired depth; b) desired shape; and c) desired effective diameter of the bore/receptacle 92, 94 in the respective bone 10, 12.

Depending upon the bone types and condition, establishing and verifying dimensional parameters for receptacles for implants is important to provide angular stability between bone parts 10, 12 after fusion and to prevent angular play between bone parts that might lead to future complications.

It is noted that the implant 72, as shown in FIGS. 18-30, has one shape with a stepped diameter, the implant 72′ in FIGS. 31-35 has a substantially uniform diameter, and the implant 72″ in FIGS. 47-61 has a still different configuration with a stepped diameter.

The implant 72′″ has a generally rectangular cross-sectional configuration, as viewed along its length, and a body that tapers in width towards each of the ends 76′″, 78′″ thereof, that are rounded to facilitate insertion into bones without hangup.

The rectangular shape produces side “voids” to accommodate the drawing of the component/suture 82 with the components in the FIG. 54 state. Other shapes to form “voids” are contemplated.

The foregoing disclosure of specific embodiments is intended to be illustrative of the broad concepts comprehended by the invention.

1. A method of fusing first and second bones at a joint, the second bone having first and second ends, a first end of the first bone at the first end of the second bone where the joint is formed, the method comprising the steps of:
obtaining an implant having a length between first and second ends;
directing the implant into an intramedullary space of the first bone so that a first length portion of the implant resides within the intramedullary space of the first bone;
exerting a force on the implant through an opening at the second end of the second bone as an incident of which: a) a part of the first length portion of the implant within the intramedullary space of the first bone moves out of the intramedullary space of the first bone; and b) one of the first and second ends of the implant is caused to move one of: i) into the intramedullary space of the second bone; and ii) further within the intramedullary space of the second bone towards the second end of the second bone.

2. The method of fusing first and second bones at a joint according to claim 1 wherein the step of directing the implant comprises directing the first end of the implant into the intramedullary space of the first bone and the one of the first and second ends of the implant is the second end of the implant.

3. The method of fusing first and second bones at a joint according to claim 2 wherein the step of exerting a force on the implant comprises exerting a force through a component extending through the opening at the second end of the second bone and engaged with the implant at or adjacent the second end of the implant.

4. The method of fusing first and second bones at a joint according to claim 2 wherein the step of exerting a force on the implant comprises exerting a force through a component extending through the opening at the second end of the second bone and engaged with the implant at a location between the first and second ends of the implant.

5. The method of fusing first and second bones at a joint according to claim 2 further comprising the step of modifying at least one of the first end of the first bone and the first end of the second bone to define cooperating surfaces at the first end of the first bone and the first end of the second bone that are brought together with the first bone and second bone in a fused relationship.

6. The method of fusing first and second bones at a joint according to claim 5 wherein the step of modifying comprises causing the cooperating surfaces at the first end of the first bone and the first end of the second bone to have substantially flat apposed surface configurations with the first bone and second bone in the fused relationship.

7. The method of fusing first and second bones at a joint according to claim 2 further comprising the step of forming a receptacle for the implant in at least one of the first bone and the second bone.

8. The method of fusing first and second bones at a joint according to claim 7 wherein the step of forming a receptacle comprises forming a receptacle in each of the first bone and the second bone, whereby with the first bone and second bone in a fused relationship, one part of the implant is frictionally maintained within the receptacle formed in the first bone and another part of the implant is frictionally maintained within the receptacle formed in the second bone.

9. The method of fusing first and second bones at a joint according to claim 2 wherein the step of exerting a force comprises exerting a force through a component made up of a suture that is connected to the implant and projects from the opening at the second end of the second bone.

10. The method of fusing first and second bones at a joint according to claim 9 wherein the suture connects to the implant at a location between the first and second ends of the implant.

11. The method of fusing first and second bones at a joint according to claim 9 further comprising the step of engaging the suture with the implant with the suture extending from the implant through the intramedullary space of the second bone so that a graspable part of the suture projects from the intramedullary space of the second bone through the opening at the second end of the second bone, and the step of exerting a force comprises exerting a force on the graspable part of the suture.

12. The method of fusing first and second bones at a joint according to claim 11 further comprising the steps of obtaining a suture engaging tool, directing a part of the suture engaging tool in a first direction through the opening at the second end of the second bone and the intramedullary space of the second bone and engaging the part of the suture engaging tool with the suture, and with the part of the suture engaging tool engaged with the suture moving the part of the suture engaging tool oppositely to the first direction to cause the graspable part of the suture to project from the intramedullary space of the second bone through the opening at the second end of the second bone.

13. The method of fusing first and second bones at a joint according to claim 11 further comprising the steps of obtaining a suture engaging tool, engaging the suture engaging tool with the suture, and directing the suture engaging tool through an opening at the first end of the second bone to thereby advance a part of the suture through the intramedullary space of the second bone to cause the graspable part of the suture to project from the intramedullary space of the second bone at the second end of the second bone.

14. The method of fusing first and second bones at a joint according to claim 9 wherein the suture is connected to the implant by directing the suture through an opening in the implant and doubling the suture against itself to create a bight portion that bears against the implant as the force is exerted through the suture.

15. The method of fusing first and second bones at a joint according to claim 2 wherein the step of exerting a force comprises exerting a force through a drawing component that is extended through the opening at the second end of the second bone and the intramedullary space of the second bone and connected to the implant at or adjacent to the second end of the implant.

16. The method of fusing first and second bones at a joint according to claim 15 wherein the drawing component is releasably connected to the implant through a connector on the drawing component that is releasably connected to a connector on the implant.

17. The method of fusing first and second bones at a joint according to claim 16 wherein the connectors on the drawing component and implant are releasably threadably engaged.

18. The method of fusing first and second bones at a joint according to claim 8 wherein the receptacle in the first bone is a bore in the first bone enlarging the intramedullary space of the first bone and the receptacle in the second bone is a bore in the second bone enlarging the intramedullary space of the second bone.

19. The method of fusing first and second bones at a joint according to claim 18 wherein with the first bone and second bone in a fused relationship an axis of the bore in the first bone is aligned with an axis of the bore in the second bone.

20. The method of fusing first and second bones at a joint according to claim 18 wherein a diameter of the bore in the first bone is different than a diameter of the bore in the second bone.

21. The method of fusing first and second bones at a joint according to claim 2 wherein the implant has a body with a non-uniform diameter between the first and second ends of the implant.

22. The method of fusing first and second bones at a joint according to claim 21 wherein the diameter of the body tapers over a length of the body.

23. The method of fusing first and second bones at a joint according to claim 2 wherein the implant has a body with a lengthwise axis and an external surface extending around the lengthwise axis, and the external surface has a non-circular shape as viewed along the lengthwise axis over at least a portion of the length of the implant, and with the first bone and second bone in a fused relationship the non-circular shape causes the implant to be keyed against turning relative to at least one of the first bone and second bone around the lengthwise axis of the body.

24. The method of fusing first and second bones at a joint according to claim 21 wherein the implant body has a first diameter over a first length portion of the body and a second diameter, different than the first diameter, over a second length portion of the body.

25. The method of fusing first and second bones at a joint according to claim 24 wherein the first length portion of the body has a first axis portion, the second length portion of the body has a second axis portion, and the first and second axes portions are coincident.

26. The method of fusing first and second bones at a joint according to claim 24 wherein the first length portion of the body has a first axis portion, the second length portion of the body has a second axis portion, and the first and second axes portions are non-coincident.

27. The method of fusing first and second bones at a joint according to claim 24 wherein the first length portion of the body has a first axis portion, the second length portion of the body has a second axis portion, and the first and second axes portions are non-parallel.

28. The method of fusing first and second bones at a joint according to claim 2 wherein the implant is made from at least one of: a) allograft bone; b) metal; c) polyetheretherketone (PEEK); d) bioabsorbable material; e) calcium salt; f) titanium; g) stainless steel; h) polyetherketoneketone (PEKK); and i) carbon fiber.

29. The method of fusing first and second bones at a joint according to claim 9 further comprising the step of separating the suture from the implant after exerting the force through the component.

30. The method of fusing first and second bones at a joint according to claim 1 wherein the implant is a first implant and further comprising the steps of obtaining a second implant and engaging the second implant with the first and second bones.

31. The method of fusing first and second bones at a joint according to claim 30 wherein the step of engaging the second implant comprises engaging the second implant with the first and second bones so that parts of the first and second bones are urged towards each other.

32. The method of fusing first and second bones at a joint according to claim 31 wherein the second implant is made up of a suture.

33. The method of fusing first and second bones at a joint according to claim 32 wherein the step of engaging the second implant with the first and second bones comprises forming the suture into a loop acting against parts of the first and second bones.

34. The method of fusing first and second bones at a joint according to claim 33 wherein the loop is a continuous loop.

35. The method of fusing first and second bones at a joint according to claim 34 wherein the loop is in the form of a box.

36. The method of fusing first and second bones at a joint according to claim 34 wherein the loop is in the form of a figure eight.

37. The method of fusing first and second bones at a joint according to claim 35 wherein the loop is additionally in the form of a figure eight.

38. The method of fusing first and second bones at a joint according to claim 7 further comprising the steps of obtaining a sound and directing a part of the sound into the formed receptacle on at least one of the first and second bones to check for at least one of: a) a desired depth; b) desired shape; and c) desired effective diameter of the formed receptacle.

39. A kit usable for fusing first and second bones at a joint formed between a first end of the first bone and a first end of the second bone, the kit comprising:
an implant having a body and a length between first and second ends;
at least one tool configured to form: a) a first receptacle by modifying an intramedullary space of the first bone; and b) a second receptacle by modifying an intramedullary space of the second bone; and
a drawing component configured to releasably engage and exert a force on the implant body with the first end of the first bone against or adjacent to the first end of the second bone and a length portion of the implant body extended into the first receptacle on the first bone so as to thereby: a) move a part of the length portion of the implant body out of the first receptacle; and b) move one of the first and second ends of the implant body one of: i) into the second receptacle; and ii) further within the second receptacle.

40. The kit usable for fusing first and second bones at a joint according to claim 39 wherein the drawing component comprises a suture and the implant has an opening through which the suture can be directed to allow the suture to be doubled against itself to define a bight portion that can be drawn against the implant body.

41. The kit usable for fusing first and second bones at a joint according to claim 40 in combination with a suture engaging tool that has a part configured to be directed through the second receptacle in a first direction to engage the suture and thereafter be moved oppositely to the first direction to be withdrawn from the second receptacle with the engaged suture to thereby expose a graspable part of the suture.

42. The kit usable for fusing first and second bones at a joint according to claim 39 wherein the drawing component has a connector that can be releasably engaged with a connector on the implant.

43. The kit usable for fusing first and second bones at a joint according to claim 42 wherein the connectors on the drawing component and implant are releasably threadably engaged.

44. The kit usable for fusing first and second bones at a joint according to claim 40 wherein the opening in the implant is at a location between the first and second ends of the implant.

45. The kit usable for fusing first and second bones at a joint according to claim 39 wherein the implant body has a non-uniform diameter between the first and second ends of the implant.

46. The kit usable for fusing first and second bones at a joint according to claim 39 wherein the implant has a body with a lengthwise axis and an external surface extending around the lengthwise axis and the external surface has a non-circular shape as viewed along the lengthwise axis over at least a portion of the length of the implant and with the first bone and second bone in a fused relationship the non-circular shape of the external surface causes the implant to be keyed against turning relative to at least one of the first bone and second bones around the lengthwise axis of the body.

47. The kit usable for fusing first and second bones at a joint according to claim 39 wherein the implant body has a first diameter over a first length portion of the implant body and a second diameter, different than the first diameter, over a second length portion of the implant body.

48. The kit usable for fusing first and second bones at a joint according to claim 39 wherein a first length portion of the implant body has a first axis portion, a second length portion of the implant body has a second axis portion, and the first and second axes portions are coincident.

49. The kit usable for fusing first and second bones at a joint according to claim 39 wherein a first length portion of the implant body has a first axis portion, a second length portion of the implant body has a second axis portion, and the first and second axes portions are non-coincident.

50. The kit usable for fusing first and second bones at a joint according to claim 39 wherein a first length portion of the implant body has a first axis portion, a second length portion of the implant body has a second axis portion, and the first and second axes portions are non-parallel.

51. The kit usable for fusing first and second bones at a joint according to claim 39 wherein the implant is made from at least one of: a) allograft bone; b) metal; c) polyetheretherketone (PEEK); d) bioabsorbable material; e) calcium salt; f) titanium; g) stainless steel; h) polyetherketoneketone (PEKK); and i) carbon fiber.

52. The kit usable for fusing first and second bones at a joint according to claim 39 further in combination with at least one sound having a projecting length configured to be extended into at least one of the first and second receptacles to thereby predetermine a relationship between the at least one of the first and second receptacles and a part or parts of the implant that is/are directed into the at least one of the first and second receptacles.

53. The kit usable for fusing first and second bones at a joint according to claim 39 wherein the implant is a first implant and further in combination with a second implant comprising a suture.

54. The kit usable for fusing first and second bones at a joint according to claim 53 wherein the suture is an osseous suture.

55. The kit usable for fusing first and second bones at a joint according to claim 39 wherein the drawing component comprises a suture and further in combination with a suture engaging tool that has a suture engaging part that is configured to engage the suture and be advanced through the second receptacle so that a part of the suture is advanced through and out of the second receptacle.

56. The kit usable for fusing first and second bones at a joint according to claim 55 wherein the suture engaging tool comprises a cutting tool to modify the intramedullary space of at least one of the first and second bones to allow the cutting tool to be advanced fully through the at least one of the first and second bones.

57. The kit usable for fusing first and second bones at a joint according to claim 56 wherein the suture engaging tool has a body and the suture engaging part and cutting tool are on the body of the suture engaging tool.

58. The kit usable for fusing first and second bones at a joint according to claim 57 wherein the body of the suture engaging tool has a length between spaced ends, and the suture engaging part and cutting tool are spaced from each other lengthwise of the body of the suture engaging tool.

59. The kit usable for fusing first and second bones at a joint according to claim 58 wherein the suture engaging part is adjacent one of the spaced ends of the body of the suture engaging tool and the cutting tool is adjacent the other of the spaced ends of the body of the suture engaging tool.
